(19) United States
(12) Patent Application Publication
(10) Pub. No.: US 2010/0324060 A1
(43) Pub. Date: Dec. 23, 2010

(54) PHARMACEUTICAL COMPOSITIONS USEFUL FOR TREATING HCV

(75) Inventors: William E. Delaney, Delaney, CA (US); Hongmei Mo, Palo Alto, CA (US); Weidong Zhong, San Ramon, CA (US)

Correspondence Address: GILEAD SCIENCES INC, 333 LAKESIDE DR, FOSTER CITY, CA, 94404, US

(73) Assignee: Gilead Sciences, Inc., Foster City, CA (US)

(21) Appl. No.: 12/818,421

(22) Filed: Jun. 18, 2010

Related U.S. Application Data
(60) Provisional application No. 61/219,654, filed on Jun. 23, 2009.

(51) Int. Cl. A61K 31/501; A61P 31/14

(52) U.S. Cl. 514/252.04

(57) ABSTRACT

This invention relates to combinations of Compound 1 and Compound 2 which are useful for treating hepatitis C virus infection.

### CROSS-REFERENCE TO RELATED APPLICATIONS

This application is filed under 35 U.S.C. 111(a) claiming the benefit under 35 U.S.C. 119(e) of U.S. Provisional Patent Application 61/219,654, filed Jun. 23, 2009 which is herein incorporated by reference in its entirety for all purposes.

### FIELD OF THE INVENTION

This invention relates to combinations of therapeutic molecules useful for treating hepatitis C virus infection.

### BACKGROUND OF THE INVENTION

Hepatitis C is recognized as a chronic viral disease of the liver which is characterized by liver disease. Although drugs targeting the liver are in wide use and have shown effectiveness, toxicity and other side effects have limited their usefulness. Inhibitors of HCV are useful to limit the establishment and progression of infection by HCV as well as in diagnostic assays for HCV.

There is a need for new HCV therapeutic agents.

### SUMMARY OF THE INVENTION

In one aspect, the present invention provides compositions that each include Compound 1 and Compound 2. Compound 1 has the following structure identified as Formula 1:

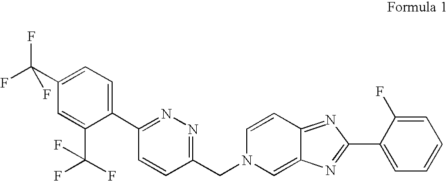

Compound 2 has the following structure identified as Formula 2:

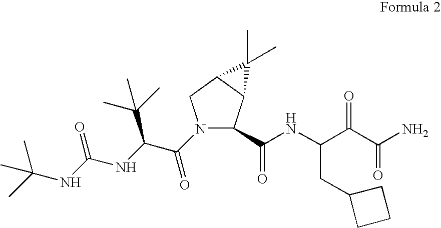

Both Compound 1 and Compound 2 inhibit HCV replication. While not wishing to be bound by theory, it is believed that Compound 1 is an inhibitor of the HCV NS5B polymerase enzyme. Compound 1 is disclosed in PCT/US2007/015553 (WO 2008/005519), which is incorporated herein by reference in its entirety. Again, while not wishing to be bound by theory, it is believed that Compound 2 is an inhibitor of the HCV NS3/4A protease enzyme. Compound 2 is disclosed in WO 2002/08244 and in WO 2003/062265, which are incorporated herein by reference in their entirety. Compound 2 has the USAN name boceprevir. The present inventors have shown the anti-HCV activity of the combination of Compound 1 and Compound 2, as described in Example 3. The data disclosed in Example 3 of the present application therefore suggests the beneficial combination of Compounds 1 and 2 as a treatment for HCV infection.

The compositions of the present invention typically include sufficient Compound 1 and Compound 2 to provide a dose of these two compounds that is effective to treat HCV infection when administered to a human being as part of a treatment regime. Thus, in one aspect, the present invention provides pharmaceutical compositions that include Compound 1 and Compound 2 and one or more pharmaceutically acceptable carrier.

For example, compositions of the present invention can include from 1 mg to 100 mg of Compound 1, and from 100 mg to 1200 mg of Compound 2. Again by way of example, compositions of the present invention can include from 30 mg to 50 mg of Compound 1, and from 600 mg to 1000 mg of Compound 2.

In another aspect, the present invention provides methods for the treatment of HCV infection in a human being, wherein each method includes the step of administering a therapeutically effective amount of a combination of Compound 1 and Compound 2 to a human being in need thereof, such as a human being infected with the hepatitis C virus. In the practice of the methods of this aspect of the invention, the combined amount of Compound 1 and Compound 2 is effective to treat HCV infection, although the amounts of Compound 1 and Compound 2 may also be individually effective to treat HCV infection. Compound 1 and Compound 2 may be administered together (e.g., in the form of a unit dosage, such as a tablet), or Compound 1 and Compound 2 may be administered separately. Compound 1 may be administered at the same time as Compound 2, or before or after the administration of Compound 2.

Typically, Compound 1 and Compound 2 are administered daily. In one embodiment, a daily dosage is administered in separate sub-doses, such as twice daily or three times per day (e.g., every eight hours during each twenty four hour period). By way of example, in the practice of the methods of this aspect of the invention, an amount of from 1 mg to 100 mg of Compound 1 and from 100 mg to 1200 mg of Compound 2 can be administered daily to a human being in need thereof. Again by way of example, an amount of from 30 mg to 50 mg of Compound 1 and from 600 mg to 1000 mg of Compound 2 can be administered daily to a human being in need thereof. The course of treatment can extend, for example, from 12 weeks to 48 weeks, such as from 12 weeks to 24 weeks.

In one embodiment, Compound 1 and Compound 2 are administered orally (e.g., in the form of a tablet or capsule). In another embodiment, Compound 1 and Compound 2 are administered by injection, such as by intravenous injection. In another embodiment, Compound 1 and Compound 2 are administered by aerosol delivery.

Another aspect of the present invention includes the use of the combination of Compound 1 and Compound 2 in the manufacture of a medicament for the treatment of HCV infection in a human being.

Another aspect of the present invention includes a composition comprising Compound 1 and Compound 2 for use in the treatment or prevention of HCV infection in a human being.

The scope of the present invention includes all combinations of aspects and embodiments.

### DETAILED DESCRIPTION

Reference will now be made in detail to certain claims of the invention, examples of which are illustrated in the accompanying structures and formulas. While the invention will be described in conjunction with the enumerated claims, it will be understood that they are not intended to limit the invention to those claims. On the contrary, the invention is intended to cover all alternatives, modifications, and equivalents, which may be included within the scope of the present invention as defined by the claims.

All documents cited herein are each incorporated by reference in their entirety for all purposes.

When trade names are used herein, applicants intend to independently include the tradename product and the active pharmaceutical ingredient(s) of the tradename product.

As used herein, “Compound 1” means Compound 1 or a pharmaceutically acceptable salt, solvate, ester or stereoisomer thereof.

As used herein, “Compound 2” means Compound 2 or a pharmaceutically acceptable salt, solvate, ester or stereoisomer thereof.

As used herein, the term “therapeutically effective amount” refers to an amount of the combination of Compound 1 and Compound 2 that is effective to ameliorate at least one symptom of HCV infection in a human being. Thus, for example, in some HCV infected individuals a therapeutically effective amount of the combination of Compound 1 and Compound 2 is effective to reduce by a statistically significant amount the viral load of HCV viral particles present in the body of the infected person. Viral load can be measured, for example, by measuring plasma HCV RNA levels using, for example, the COBAS TaqMan HCV assay (Roche Molecular Systems). Typically, an HCV infected person who is treated with the combination of Compound 1 and Compound 2 in accordance with the present invention experiences an improvement in one or all of the symptoms associated with the HCV infection. For example, an HCV patient may experience an improvement in one or all of the following symptoms that can be associated with HCV infection: fever, headache, muscle aches, fatigue, loss of appetite, nausea, vomiting and diarrhea.

The present invention relates to methods, uses, and compositions comprising Compound 1 and Compound 2. Compound 1 has the following structure:

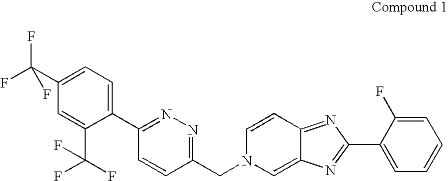

Compound 2 has the following structure:

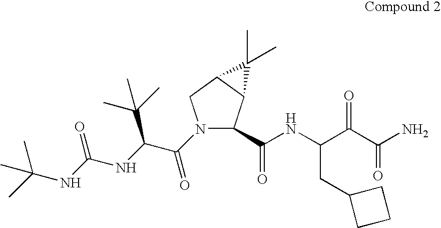

The data set forth in Example 3 show that the combination of Compound 1 and Compound 2 has anti-HCV activity, as measured in an in vitro HCV replicon assay, and that Compound 1 and Compound 2 interact synergistically. Thus, the combination of Compound 1 and Compound 2 is useful, for example, for inhibiting HCV replication in vitro and in vivo, such as inhibiting HCV replication in human beings infected with HCV. Compounds 1 and 2, and the combination thereof, can also be used, for example, in assays to identify additional molecules that inhibit, or otherwise affect, HCV replication in vitro or in vivo, or to study the mechanism of HCV replication in living cells.

Salt forms, or solvates, of Compounds 1 and 2 can be used in the practice of the present invention. Typically, but not necessarily, the salts of Compounds 1 and 2 are pharmaceutically acceptable salts. Salts encompassed within the term “pharmaceutically acceptable salts” refer to non-toxic salts of Compounds 1 and 2.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

The pharmaceutical compositions of the present invention include Compound 1 and Compound 2 in the pure state or in the form of a composition in which the compounds are combined with any other pharmaceutically compatible substance, which can be inert or physiologically active. The resulting pharmaceutical compositions can be used, for example, to treat HCV infection in a human being.

The manner in which Compounds 1 and 2 are administered can vary. For example, the compositions may be administered orally, such as in liquid form within a solvent such as an aqueous or non-aqueous liquid, or within a solid carrier. Compositions for oral administration include pills, tablets, capsules, caplets, syrups, and solutions, including hard gelatin capsules and time-release capsules. Standard excipients include binders, fillers, colorants, solubilizers and the like. Compositions can be formulated in unit dose form, or in multiple or subunit doses. Compositions including a liquid pharmaceutically inert carrier such as water or other pharmaceutically compatible liquids or semisolids can be used. The use of such liquids and semisolids is well known to those of skill in the art.

Again by way of example, the compositions can be administered via injection, i.e., intravenously, intramuscularly, subcutaneously, intraperitoneally, intraarterially, intrathecally; and intracerebroventricularly. Intravenous administration is the preferred method of injection. Suitable carriers for injection are well known to those of skill in the art and include, for example, 5% dextrose solutions, saline, and phosphate-buffered saline. The compounds can also be administered as an infusion or injection, namely, as a suspension or as an emulsion in a pharmaceutically acceptable liquid or mixture of liquids.

The compounds can also be administered directly to the respiratory tract by inhalation, namely, in the form of an aerosol either nasally or orally. Thus, one aspect of the present invention includes a novel, efficacious, safe, nonirritating, and physiologically compatible inhalable composition comprising Compound 1 and Compound 2 which are useful for treating HCV infection.

Other examples of delivery routes for Compounds 1 and 2 include rectal delivery, such as by the administration of a suppository, or transdermal administration.

Compounds 1 and 2 may be administered together or separately and, when administered separately, administration may occur simultaneously or sequentially, in any order. The amounts of Compounds 1 and 2, and the relative timings of administration, will be selected in order to achieve the desired therapeutic effect. The administration of Compound 1 and Compound 2 may be in combination by administration concomitantly in: (1) a unitary pharmaceutical composition including both compounds; or (2) separate pharmaceutical compositions each including one of the compounds. The combination may also be administered separately in a sequential manner wherein one treatment agent is administered first and the other second or vice versa. Such sequential administration may be close in time or remote in time.

Compounds 1 and 2 are typically administered in the form of pharmaceutical compositions that include at least one pharmaceutically acceptable carrier. The term “pharmaceutically acceptable carrier” as used herein means any material or substance formulated with the active ingredient in order to facilitate its preparation and/or its application or dissemination to the site to be treated. Suitable pharmaceutical carriers for use in the compositions of this invention are well known to those skilled in the art. They include additives such as wetting agents, dispersing agents, adhesives, emulsifying agents, solvents, glidants, coatings, antibacterial and antifungal agents (for example phenol, sorbic acid, chlorobutanol), and isotonic agents (such as sugars or sodium chloride), provided that the same are consistent with pharmaceutical practice, i.e. they are not toxic to mammals.

The pharmaceutical compositions of the present invention are prepared in any known manner, for instance by homogeneously mixing, coating and/or grinding the active ingredients in a one-step or multi-step procedure, with the selected carrier material and, where appropriate, other additives such as surface-active agents.

The pharmaceutical compositions of the present invention can include solubilized forms of Compound 1 and Compound 2, where Compounds 1 and 2 are dissolved in an appropriate solvent or solubilizing agent, or combinations thereof. The solvent typically includes various organic acids (typically C4-C24) such as capric, oleic or lauric acid. In addition, polyethylene glycols (PEGs) and/or short, medium, or long chain mono, di, or triglycerides can be employed to dissolve Compounds 1 and 2 for use in a liquid formulation. Pegylated short, medium or long chain fatty acids may also be used. Typically, the preparation is aqueous, i.e, water is the only solvent per se although it generally will also include the solubilizing agent such as an organic acid or the other agents described above.

The most common organic acids are the carboxylic acids whose acidity is associated with the carboxyl group —COOH. Sulfonic acids, containing the group OSO₃H, are relatively stronger acids for use herein. In general, the acid desirably contains a lipophilic domain. Mono- or di-carboxylic acids are suitable.

Suitable surface-active agents optionally are used with any of the pharmaceutical compositions of this invention. Such agents also are known as emulgents or emulsifiers, and are useful in the pharmaceutical compositions of the present invention. They are non-ionic, cationic and/or anionic materials having suitable emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable from coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcoholamine salts of dodecylbenzene sulphonic acid or dibutyl-naphthalenesulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidyl-choline, dipalmitoylphoshatidyl-choline and their mixtures. Aqueous emulsions with such agents are within the scope of this invention.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8-C22 alkyl radical (e.g. cetyl, lauryl, palmityl, myristyl and oleyl) and, as further substituents, unsubstituted or halogenated lower alkyl, benzyl and/or hydroxy-lower alkyl radicals.

A more detailed description of surface-active agents suitable for this purpose is found in “McCutcheon's Detergents and Emulsifiers Annual” (MC Publishing Crop., Ridgewood, N.J., 1981), “Tensid-Taschenbucw”, 2nd ed. (Hanser Verlag, Vienna, 1981) and “Encyclopaedia of Surfactants,” (Chemical Publishing Co., New York, 1981). Further details on techniques for formulation and administration may be found in the latest edition of Remington's Pharmaceutical Sciences (Maack Publishing Co, Easton, Pa.).

Compositions comprising Compound 1 and Compound 2 may be manufactured in a manner similar to that known in the art (e.g., by means of conventional mixing, dissolving, granulating, dragee-making, levigating, emulsifying, encapsulating, entrapping, or lyophilising processes). Compositions comprising Compound 1 and Compound 2 may also be modified to provide appropriate release characteristics, e.g., sustained release or targeted release, by conventional means (e.g., coating).

In another aspect, the present invention provides methods for the treatment of HCV infection in a human being, wherein each method includes the step of administering a therapeutically effective amount of a combination of Compound 1 and Compound 2 to a human being in need thereof, such as a human being infected with the hepatitis C virus. The pharmaceutical compositions of the present invention are useful, for example, in the practice of the treatment methods of the present invention. In the practice of the methods of this aspect of the invention, the combined amount of Compound 1 and Compound 2 is effective to treat HCV infection, although the amounts of Compound 1 and Compound 2 may also be individually effective to treat HCV infection. Compound 1 and Compound 2 may be administered together (e.g., in the form of a unit dosage, such as a tablet), or Compound 1 and Compound 2 may be administered separately. Compound 1 may be administered at the same time as Compound 2, or before or after the administration of Compound 2.

Typically, Compound 1 and Compound 2 are administered daily. In one embodiment, a daily dosage is administered in separate sub-doses, such as twice daily or three times per day (e.g., every eight hours during each twenty four hour period). By way of example, in the practice of the methods of this aspect of the invention, an amount of from 1 mg to 100 mg of Compound 1 and from 100 mg to 1200 mg of Compound 2 can be administered daily to a human being in need thereof. Again by way of example, an amount of from 30 mg to 50 mg of Compound 1 and from 600 mg to 1000 mg of Compound 2 can be administered daily to a human being in need thereof. The course of treatment can extend, for example, from 12 weeks to 48 weeks, such as from 12 weeks to 24 weeks.

The following examples merely illustrate the best mode now contemplated for practicing the invention, but should not be construed to limit the invention.

### Example 1a

### Synthesis of 5-({6-[2,4-bis(trifluoromethyl)phenyl]pyridazin-3-yl}methyl)-2-(2-fluorophenyl)-5H-imidazo[4,5-c]pyridine

Compound 1 has the IUPAC name: 5-({6-[2,4-bis(trifluoromethyl)phenyl]pyridazin-3-yl}methyl)-2-(2-fluorophenyl)-5H-imidazo[4,5-c]pyridine, and the CAS name: 5H-imidazo[4,5-c]pyridine, 5-[[6-[2,4-bis(trifluoromethyl)phenyl]pyridazin-3-yl]methyl]-2-(2-fluorophenyl).

In this method for making Compound 1, dimethoxyethane or its related solvents, all having the general formula R¹OR²O(R⁴O)ₐR³ wherein each of R¹, R², R³ and R⁴ are independently selected from C₁-C₆ alkyl and a is 0 or 1, have been found to be particularly advantageous over the conventional solvent DMF. Typically, each of R¹, R², R³ and R⁴ are independently C₁-C₂ alkyl and usually a is 0. C₁-C₆ alkyl includes fully saturated primary, secondary or tertiary hydrocarbon groups with 1 to 6 carbon atoms and thereby includes, but is not limited to methyl, ethyl, propyl, butyl, etc.

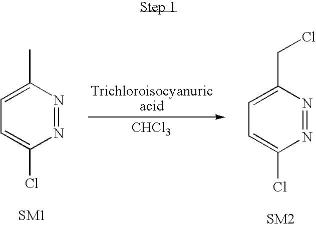

| Compound | MW | Amount | mmoles | Equivalents |
|---|---|---|---|---|
| SM1 | 128.56 | 5 g | 38.9 | 1 |
| TCCA | 232.41 | 3.62 g | 15.6 | 0.4 |
| CHCl₃ | | 130 mL | | |

To a solution of the commercially available starting material (SM1) in CHCl₃, trichloroisocyanuric acid (TCCA) was added at 60° C. Then the solution was stirred for 1.5 hrs., cooled down and filtered with HiFlo-Celite. The filtrate was concentrated and dried with vacuum. The yield was 5.037 g of SM2.

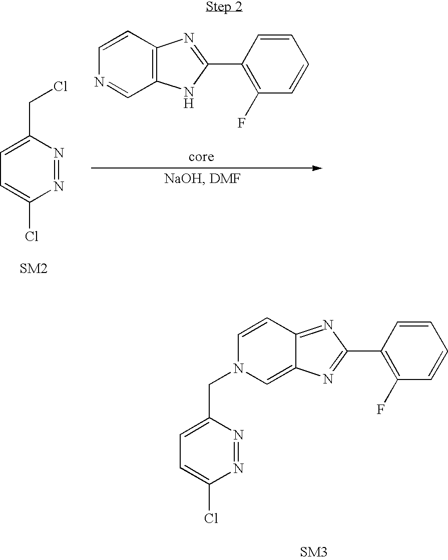

| Compound | MW | Amount | mmoles | Equivalents |
|---|---|---|---|---|
| SM2 | 163 | 5.073 g | 31.12 | 1 |
| Core | 213.2 | 6.635 g | 31.12 | 1 |
| NaOH (10%) | 40 | 1.245 g | 31.12 | 1 |
| DMF | | 320 mL | | |

To a solution of the starting material designated as “core” (obtained as described below) in DMF (dimethylformamide), NaOH was added. Then SM2 (obtained from step 1) was dissolved in DMF (20 mL) and added to the solution slowly. The reaction was stirred for 3 hrs, was diluted with water and extracted with EtOAc. The organic layer was dried with Na₂SO₄. The solvent was removed and the product recrystallized with DCM (dichloromethane). The yield was 51 g of SM3.

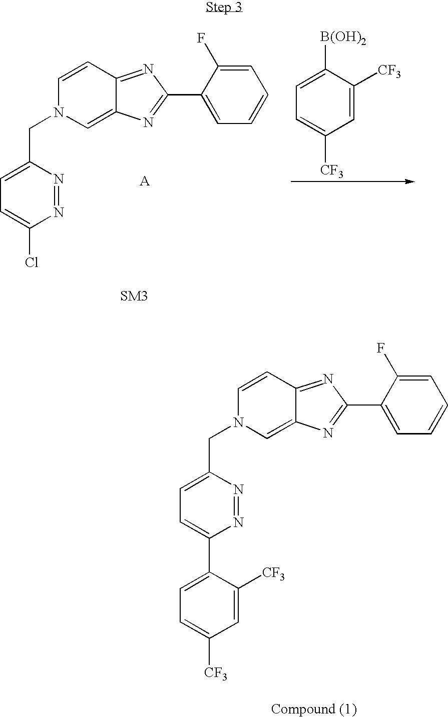

| Compound | MW | Amount | Moles | Equivalents |
|---|---|---|---|---|
| SM3 | 453.79 | 95 mg | 0.209 | 1 |
| DME | 500 μL | | | |
| 2N aq. Na₂CO₃ | | 313 μL | 0.626 | 3 |
| 2,4-bisCF₃- | 257.93 | 80.9 mg | 0.313 | 1.5 |
| phenylboronic | | | | |
| acid | | | | |
| Pd(PPh₃)₄ | 1155 | 12 mg | 0.0104 | 0.05 |

The compound designated as “SM3” was dissolved in dimethoxyethane (DME). To this solution was added 2,4-bis(trifluoromethyl)phenylboronic acid and a 2N aq. Na₂CO₃ solution. To the resulting biphasic mixture was added Pd(PPh₃)₄ and the reaction was then heated at 80° C. for 72 hrs. The reaction was cooled to room temperature and filtered through Celite and the Celite washed with EtOAc. The filtrate was concentrated in vacuo. The residue was purified on 6 g SiO2 using MeOH/CH₂Cl₂ to elute compound. The compound thus obtained was contaminated with PPh₃(O). The product was repurified on a 1 mm Chromatotron plate with 0 to 5% MeOH/CH₂Cl₂ in 1% steps. The pure fractions were combined and concentrated in vacuo, then dried, on high vacuum for 12 hrs. 11.8 mg of the free base of compound (1) was obtained with no PPh₃ contamination.

¹H NMR (300 MHz, CD₃OD)

6.20 (s, 2)

7.32 (m, 3)

7.52 (m, 1)

7.78 (d, 1)

7.89 (d, 1)

7.95 (s, 2)

8.15 (m, 3)

8.35 (d, 1)

9.12 (s, 1)

LC/MS M+H=518

### Example 1b

### Synthesis of 5-({6-[2,4-bis(trifluoromethyl)phenyl]pyridazin-3-yl}methyl)-2-(2-fluorophenyl)-5H-imidazo[4,5-c]pyridine

This example is directed to an additional method for making compound (1), employing the following schemes.

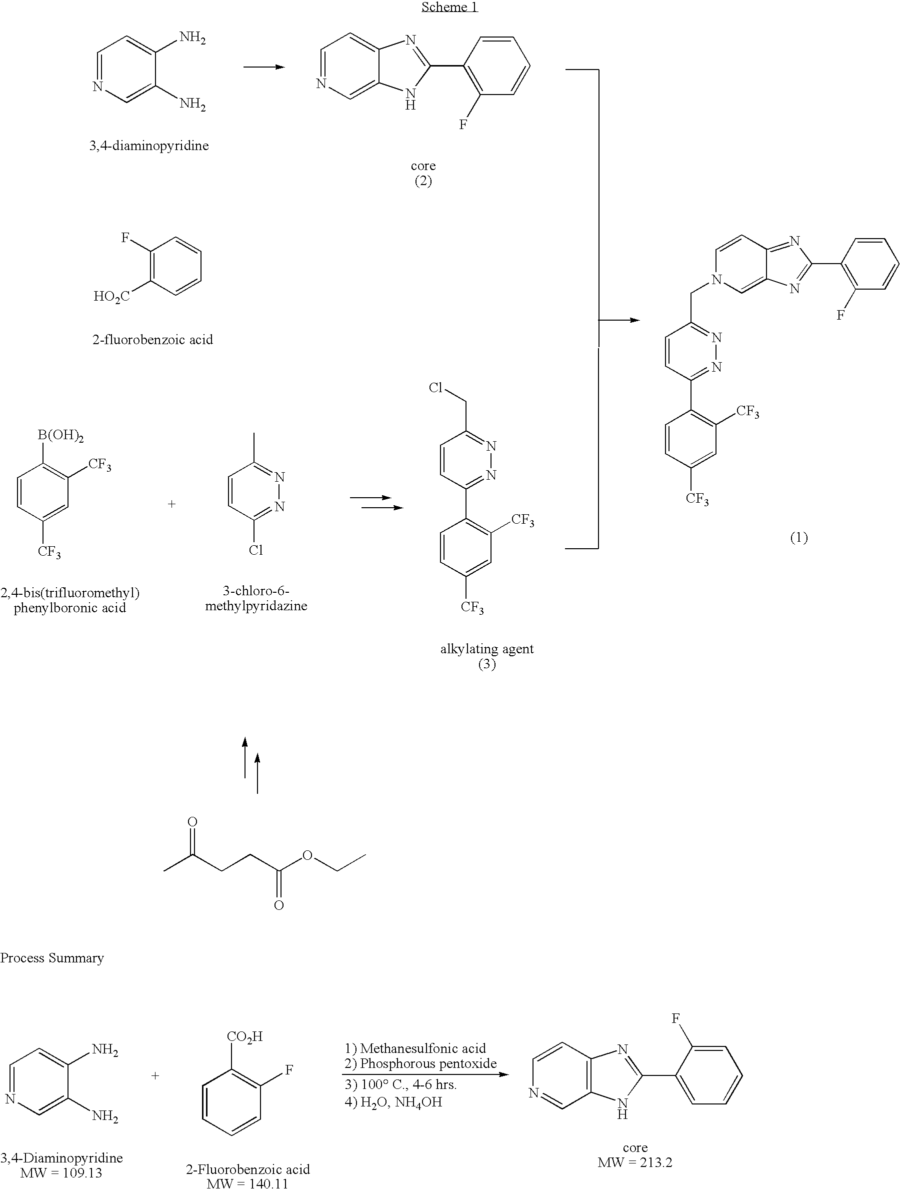

Methanesulfonic acid was added to 2-fluorobenzoic acid in a reactor with active cooling keeping T≦50° C. 3,4-Diaminopyridine was then added portionwise to this cooled slurry, keeping T≦35° C. The contents of the reactor were then heated to 50° C. Phosphorus pentoxide was added in a single charge. The reaction was then heated at 90-110° C. for at least 3 hours. The reaction was sampled for completion by HPLC analysis. The reaction was cooled to ambient temperature and water was added portionwise slowly to quench the reaction. The reaction was then diluted with water. In solubles were removed by filtration. The pH of the filtrate was adjusted to 5.5-5.8 with ammonium hydroxide. The reaction was allowed to self-seed and granulate for ˜4 hours at ambient temperature. The pH was then adjusted to 8.0-9.3 with ammonium hydroxide. The slurry was held at ambient temperature for at least 2 hours. The solids were isolated by filtration and washed with water, followed by IPE. The wet cake was dried in vacuo at not more than 60° C. until ≦1% water remains. The dry product is the compound designated as “core”.

| Summary of Materials | M.W. | Wt. Ratio | Mole ratio |
|---|---|---|---|
| 3,4-Diaminopyridine | 109.13 | 1.0 | 1.0 |
| 2-Fluorobenzoic acid | 140.11 | 1.4 | 1.1 |
| Methanesulfonic acid | 96.1 | 7.0 | 8.0 |
| Phosphorus pentoxide | 141.94 | 1.3 | 1.0 |
| Water | 18.02 | 40 | — |
| Isopropyl ether | 102.17 | 5.0 | — |
| Ammonium hydroxide | 35.09 | ~10 | — |

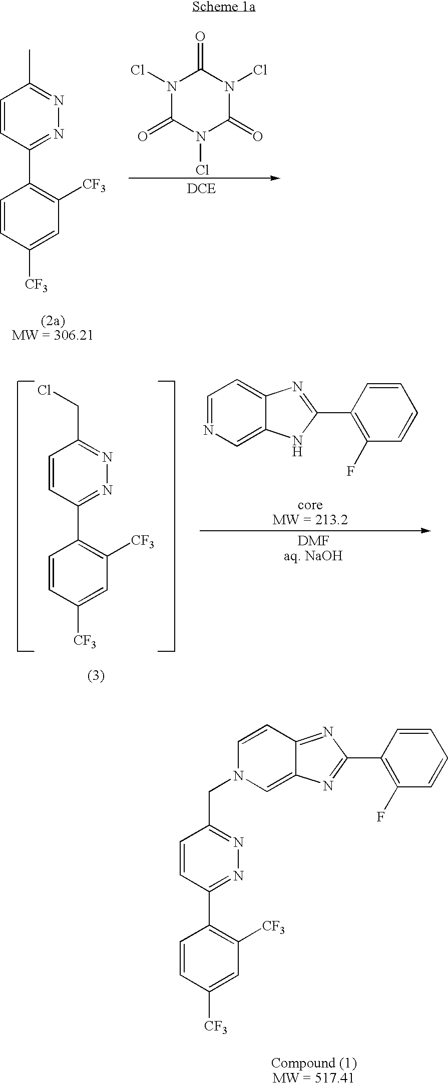

A solution of 2a in 1,2-dichloroethane was heated to 40-45° C. Trichloroisocyanuric acid was added and the mixture was heated at 60-70° C. for at least 2 hours. The reaction was sampled for completion by HPLC analysis. The reaction was cooled to ambient temperature. Celite was added to absorb insolubles, then solids were removed by filtration. The filtrate was washed with 0.5 N sodium hydroxide solution. The organic layer was concentrated to lowest stirrable volume and displaced with DMF. The compound designated as “core” and 10% aqueous sodium hydroxide solution were added. The reaction was stirred at ambient temperature for at least 8 hours. The reaction was sampled for completion by HPLC analysis. An additional 10% charge of 10% sodium hydroxide solution was added to the reaction. The reaction was then charged into water to isolate the crude product, compound (1). After granulating for at least 1 hour, the solids were isolated and washed with water and isopropyl ether. The wet cake was recrystallized from ethyl acetate to afford low melt (˜220° C.) Compound (1) (polymorph I). The wet-cake was then reslurried in ethyl acetate in the presence of less than about 0.5% water to obtain the high melt (˜236° C.) Compound (1) (polymorph II). The solids were collected by filtration and washed with ethyl acetate. The wet cake was dried in vacuo at not more than 60° C. to obtain the dry crystalline polymorph II.

| Summary of Materials | M.W. | Wt. Ratio | Mole ratio |
|---|---|---|---|
| 3-chloro-6-methylpyridazine | 128.56 | 1.0 | 1.0 |
| 2,4bis(trifluromethyl)phenylboronic | 257.93 | 4.0 | 2.0 |
| acid | | | |
| X-Phos | 476.72 | 0.18 | 0.05 |
| Palladium acetate | 224.49 | 0.04 | 0.025 |
| 1,2-Dimethoxyethane | 90.12 | 16.7 | — |
| Potassium carbonate | 138.21 | 2.15 | 2.0 |
| Water | 18.02 | 7.8 | — |
| Copper iodide | 190.45 | 0.037 | 0.025 |
| Celite | — | 0.25 | — |
| Heptane | 100.2 | 22.4 | — |

### Example 2

### Preparation of Compound 2

Compound 2 has the IUPAC name: (1R,2S,5S)-3-[N-(N-tert-Butylcarbamoyl)-3-methyl-L-valyl]-N-(2-cyclobutyl-1-oxamoylethyl)-6,6-dimethyl-3-azabicyclo[3.1.0]hexane-2-carboxamide. Compound 2 can be synthesized using the following synthetic scheme.

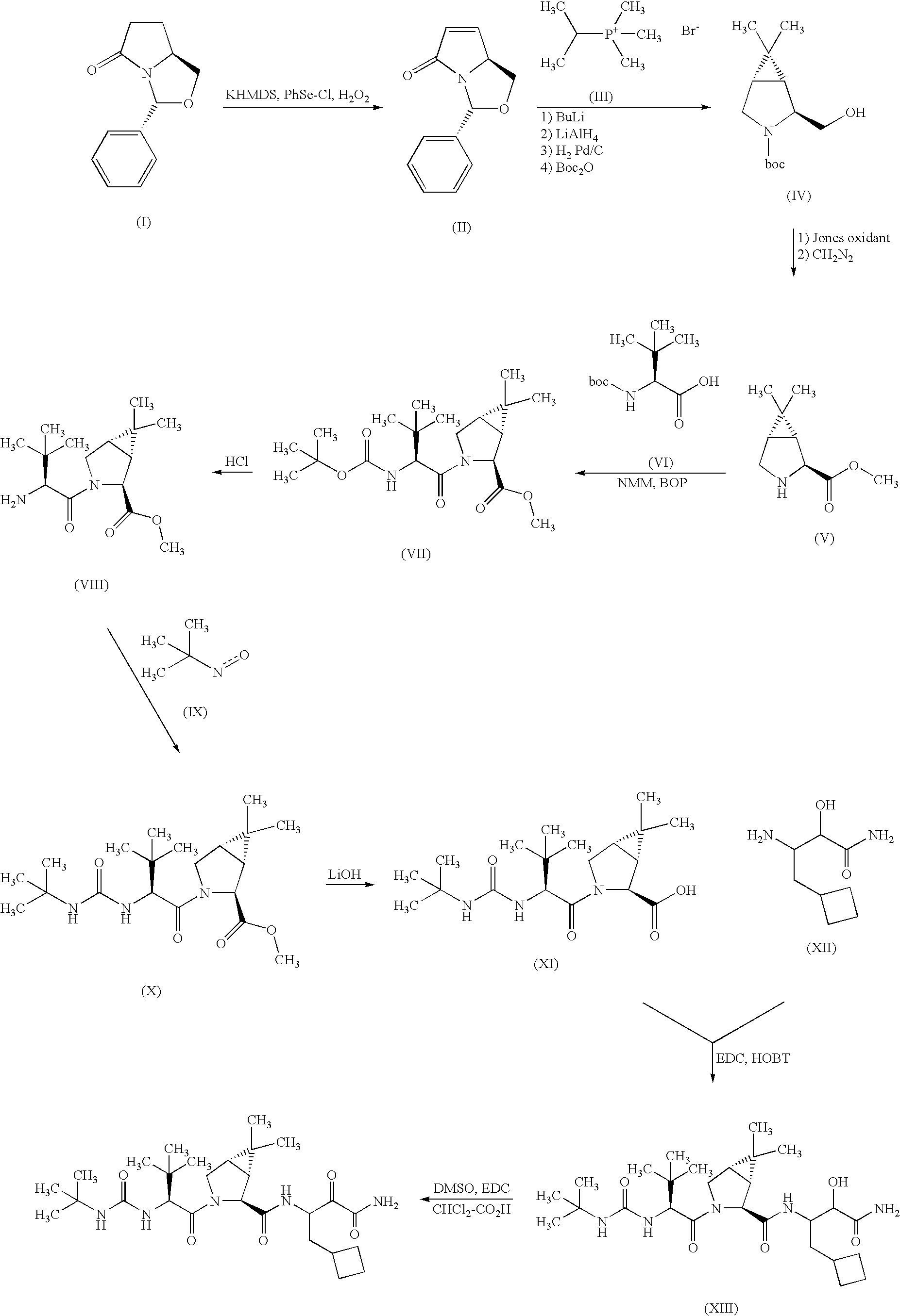

The dehydrogenation of 2-phenylperhydropyrrolo[1,2-c]oxazol-4-one (I) by means of KHMDS, Ph-Se—Cl and H₂O₂ gives 2-phenyl-4,6a-dihydro-1H-pyrrolo[1,2-c]oxazol-4-one (II), which is cyclopropanated by means of isopropyl-trimethylphosphonium bromide (III) by means of BuLi and LiAlH₄, followed by hydrogenation with H₂ over Pd/C and final N-protection with Boc₂O to yield the cyclopropa-prolinol (IV). The oxidation of (IV) with Jones reagent, followed by methylation with diazomethane affords the cyclopropa-proline methyl ester (V), which is condensed with N-Boc-tert-leucine (VI) by means of NMM and BOP to provide the dipeptide (VII). The N-Boc deprotection of (VII) by means of HCl gives the dipeptide (VIII), which is condensed with tert-butyl isocyanate (IX) in dichloromethane to yield the ureide (X). The hydrolysis of the ester group of (X) by means of LiOH in THF/water affords the acidic dipeptide (XI), which is condensed with 3-amino-4-cyclobutyl-2-hydroxybutyramide (XII) by means of EDC and HOBT to provide the alpha-hydroxyamide (XIII). Finally this compound is oxidized by means of DMSO, dichloroacetic acid and EDC in toluene to afford the target alpha-oxo-tripeptide.

The synthesis of Compound 2 is also described in S. Venkatraman et al., J. Med. Chem., 49:6074-6086 (2006) which publication is incorporated herein by reference.

### Example 3

### Anti-HCV Activity of the Combination of Compound 1 and Compound 2

### Materials and Methods.

Compound 1 was synthesized by Gilead Sciences (Foster City, Calif.). Compound 2 was purchased from Acme Bioscience, Ltd. (Belmont, Calif.).

HCV genotype 1b replicon cells (Huh-luc) were obtained from Reblikon (Mainz, Germany). The replicon in these cells is designated I389luc-ubi-neo/NS3-3′/ET and encodes a selectable resistance marker (neomycin phosphotransferase) as well as the firefly luciferase reporter gene. Huh-luc cells were maintained in Dulbecco's Modified Eagle's Medium (DMEM; GIBCO, Carlsbad, Calif.) supplemented with 10% fetal bovine serum (FBS; Hyclone, Logan, Utah) and 0.5 mg/mL of G-418 (GIBCO). Cells were passaged twice a week and maintained at subconfluent levels.

Replicon cells were seeded in 96-well plates at a density of 5×10³ cells per well in 100 μL of DMEM culture medium, excluding G-418. Compounds 1 and 2 were serially diluted 1:3 in 100% DMSO (Sigma). These serial dilutions were added to the cells at a 1:200 dilution to achieve a final concentration of 0.5% DMSO in a total volume of 200 μL. Plates were incubated at 37° C. for 3 days, after which culture media were removed and cells were lysed and assayed for luciferase activity using a commercial luciferase assay (Promega, Madison, Wis.). HCV replication levels in drug-treated samples were expressed as a percentage of those in untreated controls (defined as 100%), and data were fit to the logistic dose response equation y=a/(1+(x/b)c) using XLFit4 software (IDBS, Emeryville, Calif.). EC50 values were calculated from the resulting equations as described previously (Delaney, W. E., et al., Antimicrobial Agents Chemotherapy, 45(6):1705-1713 (2001)). Replicon cells were seeded in 96-well plates at a density of 5×103 cells per well in 100 μL of culture medium. Compounds 1 and 2 were serially diluted in 100% DMSO as described above and added in a matrix format to 96-well plates, achieving a defined set of different drug concentrations and ratios in a final volume of 200 μL and a final DMSO concentration of 0.5%. For each individual drug, the EC50 value was selected as the midpoint for the concentration range tested. Cells were incubated for three days and analyzed for luciferase expression as indicated above. For the combination study, two independent experiments were performed in triplicate.

Data were analyzed using the MacSynergy II program developed by Prichard and Shipman (Prichard M N, Aseltine K R, Shipman C, Jr., MacSynergy™ II, Version 1.0. University of Michigan, Aim Arbor, Mich., 1993; Prichard M. N., Shipman C., Jr., Antiviral Res 14 (4-5):181-205 (1990); Prichard M. N., Shipman C, Jr., Antivir Ther 1 (1):9-20 (1996); Prichard M. N., et al., Antimicrob Agents Chemother 37 (3):540-5 (1993). The software calculates theoretical inhibition assuming an additive interaction between drugs (based on the Bliss Independence model) and quantifies statistically significant differences between the theoretical and observed inhibition values. Plotting these differences in three dimensions results in a surface where elevations in the Z-plane represent antiviral synergy and depressions represent antiviral antagonism between compounds. The calculated volumes of surface deviations are expressed in nM²%. Per Prichard and Shipman, combination effects are defined as:
Highly synergistic if volumes >100 nM².
Slightly synergistic if volumes are >50 and ≦100 nM².
Additive if volumes are >−50 nM² and ≦50 nM².
Slightly antagonistic if volumes are >−100 nM² and ≦−50 nM².
Antagonistic if volumes are ≦−100 nM².

### Results.

The antiviral effect of Compound 1 in combination with Compound 2 was evaluated using the HCV 1b replicon system. The resulting data were analyzed using MacSynergy II, which provides surface plots displaying significant deviations from additivity. Quantification of statistically significant deviations from additivity from two to three independent experiments is summarized in Table 1. Compound 2 had synergy volume between 50 and 100 nM²% and antagonism volume between 0 and −25 nM²% when paired with Compound 1. These results suggest that Compound 1 has a moderate synergistic interaction with Compound 2.

TABLE 1
Quantification of Antiviral Synergy and Antagonism and
Drug Interactions for the Combination of Compound 1
and Compound 2

| Drug Used | Synergy | Antagonism | |
|---|---|---|---|
| in Combination | Volume | Volume | |
| with Compound 1 | (nM²)^{a} | (nM²)^{a} | Interaction |
| Compound 2 | 91.0 ± 17.7 | −8.0 ± 13.6 | Moderate synergy |

^{a}Values represent the mean ± standard deviation of two or three independent experiments performed in triplicate

What is claimed:

1. A composition comprising Compound 1 and Compound 2, or salts or solvates of Compound 1 and Compound 2, wherein Compound 1 has the structure shown in Formula 1 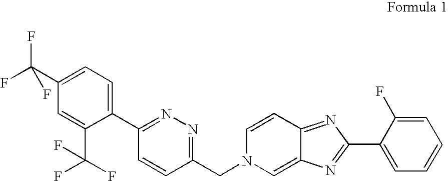
and Compound 2 has the structure shown in Formula 2

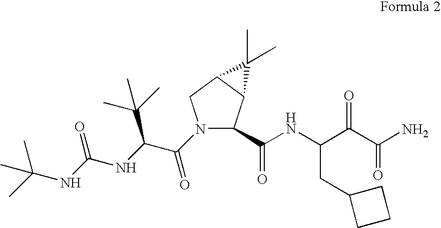

2. The composition of claim 1 wherein Compound 1 is present in an amount of from 1 mg to 100 mg.

3. The composition of claim 1 wherein Compound 1 is present in an amount of from 30 mg to 50 mg.

4. The composition of claim 1 wherein Compound 2 is present in an amount of from 100 mg to 1200 mg.

5. The composition of claim 1 wherein Compound 2 is present in an amount of from 600 mg to 1000 mg.

6. The composition of claim 1 wherein Compound 1 is present in an amount of from 1 mg to 100 mg, and Compound 2 is present in an amount of from 100 mg to 1200 mg.

7. The composition of claim 1 wherein said composition is a solid composition.

8. The composition of claim 1 wherein said composition is a liquid composition.

9. The composition of claim 1 further comprising a pharmaceutically acceptable carrier.

10. The composition of claim 9 wherein said composition is a solid composition.

11. The composition of claim 10 wherein said composition is in the form of a tablet.

12. The composition of claim 9 wherein said composition is a liquid composition.

13. The composition of claim 9 wherein Compound 1 is present in an amount of from 1 mg to 100 mg.

14. The composition of claim 9 wherein Compound 1 is present in an amount of from 30 mg to 50 mg.

15. The composition of claim 9 wherein Compound 2 is present in an amount of from 100 mg to 1200 mg.

16. The composition of claim 9 wherein Compound 2 is present in an amount of from 600 mg to 1000 mg.

17. The composition of claim 9 wherein Compound 1 is present in an amount of from 1 mg to 100 mg, and Compound 2 is present in an amount of from 100 mg to 1200 mg.

18. A method for the treatment of HCV infection in a human being, wherein the method comprises the step of administering a therapeutically effective amount of a combination of Compound 1 and Compound 2 to a human being infected with HCV.

19. The method of claim 18 wherein the combination of Compound 1 and Compound 2 comprises a daily dosage of Compound 1 of from 1 mg to 100 mg.

20. The method of claim 18 wherein the combination of Compound 1 and Compound 2 comprises a daily dosage of Compound 1 of from 30 mg to 50 mg.

21. The method of claim 18 wherein the combination of Compound 1 and Compound 2 comprises a daily dosage of Compound 2 of from 100 mg to 1200 mg.

22. The method of claim 18 wherein the combination of Compound 1 and Compound 2 comprises a daily dosage of Compound 2 of from 600 mg to 1000 mg.

23. The method of claim 18 wherein the combination of Compound 1 and Compound 2 comprises a daily dosage of Compound 1 of from 1 mg to 100 mg and a daily dosage of Compound 2 of from 100 mg to 1200 mg.

24. The method of claim 18 wherein Compound 1 is administered to the human being at the same time as Compound 2.

25. The method of claim 18 wherein Compound 1 is administered to the human being before administration of Compound 2 to the human being.

26. The method of claim 18 wherein Compound 1 is administered to the human being after administration of Compound 2 to the human being.

27. The method of claim 24 wherein Compound 1 and Compound 2 are administered to the human being in the form of a tablet.

28. The method of claim 27 wherein the tablet comprises from 1 mg to 100 mg of Compound 1 and from 100 mg to 1200 mg of Compound 2.

29. The method of claim 18 wherein Compound 1 and Compound 2 are administered once per day to the human being.

30. The method of claim 18 wherein Compound 1 and Compound 2 are administered more than once per day to the human being.

31. The method of claim 18 wherein Compound 1 and Compound 2 are administered at least once per day to the human being for a period of from 12 weeks to 48 weeks.

32. The method of claim 18 wherein Compound 1 and Compound 2 are administered orally to the human being.

33. The method of claim 18 wherein Compound 1 and Compound 2 are administered to the human being by injection.

34. Use of a combination of Compound 1 and Compound 2 in the manufacture of a medicament for the treatment of HCV infection in a human being.

35. A composition comprising Compound 1 and Compound 2 for use in the treatment of HCV infection in a human being.
